(19) United States
(12) Patent Application Publication
(10) Pub. No.: US 2025/0169973 A1
(43) Pub. Date: May 29, 2025

(54) DELIVERY CATHETER AND SYSTEM

(71) Applicant: V-FLOW 21 LTD., Modiin-Maccabim-Reut (IL)

(72) Inventor: Boaz HARARI, Ganei Tikva (IL)

(21) Appl. No.: 18/834,028

(22) PCT Filed: Feb. 2, 2023
(86) PCT No.: PCT/IL2023/050122

§ 371 Date: Jul. 29, 2024

Related U.S. Application Data
(60) Provisional application No. 63/305,782, filed on Feb. 2, 2022.

(51) Int. Cl. A61F 2/966; A61F 2/90; A61F 2/95

(52) U.S. Cl. CPC A61F 2/966; A61F 2/90; A61F 2/9517

(57) ABSTRACT

A catheter for stent delivery includes an elongated catheter body having an outer catheter tube surrounding an inner catheter tube. The elongated catheter body has a distal portion that is optionally more elastic than a proximally adjacent portion. The catheter further includes a stent holder for holding a stent within the outer catheter tube at the proximally adjacent portion, a first mechanism for advancing the stent forward into the distal portion and a second mechanism for pulling back the outer catheter tube to thereby deploy the stent.

### RELATED APPLICATION/S

This application claims the benefit of priority of U.S. Provisional Patent Application No. 63/305,782 filed on 2 Feb. 2022, the contents of which are incorporated herein by reference in their entirety.

### BACKGROUND

The present invention relates to a catheter for delivering a device through tortuous anatomy and to a stent deliverable by. Embodiments of the present invention relate to a catheter configured for delivering a deployable device such as a stent through tortuous blood vessels.

Minimally invasive diagnostic or therapeutic procedures oftentimes utilize catheters that are navigated through vessels such as blood vessels, urinary tract vessels, fallopian tubes, bile ducts, and the like.

An intravascular catheter is inserted and advanced through an introducer positioned at an access site (e.g., femoral artery) and is manually guided through the vasculature to a desired location.

Intravascular catheters must be flexible enough to navigate through tortuous vasculature without damaging tissue, yet stiff enough to provide the pushability necessary for advancement through the vasculature and support for internally guided medical devices such as stents.

To enable navigation through tortuous anatomy catheters can have a stiff main body portion and a softer distal portion. The stiff main body portion gives the catheter sufficient pushability and torqueability while the distal portion is soft enough to allow tracking over a guide wire and maneuvering through a tortuous path in the vasculature.

While such catheters can be successfully guided through tortuous anatomy to deliver therapeutics, when carrying a deployable device such as a stent, such catheters lose some of their maneuverability due to the fact that the stent is carried on the distal portion of the catheter and as such it increases the stiffness of the catheter distal portion.

There is thus a need for, and it would be highly advantageous to have, a catheter that can be easily maneuvered through tortuous anatomy devoid of the above limitations.

### SUMMARY

According to one aspect of the present invention there is provided a catheter for stent delivery comprising an elongated catheter body having an outer catheter tube surrounding an inner catheter tube, the elongated catheter body having a distal portion that is more elastic than a proximally adjacent portion of the elongated catheter body; a stent holder for holding a stent within the outer catheter tube at the proximally adjacent portion; a first mechanism for advancing the stent forward into the distal portion; and a second mechanism for pulling back the outer catheter tube to thereby deploy the stent.

According to embodiments of the present invention the inner catheter tube includes a guidewire lumen.

According to embodiments of the present invention the guidewire lumen extends from a first opening at a distal tip of the elongated catheter body to a second opening at a side wall of the outer catheter tube.

According to embodiments of the present invention the catheter further comprises a nose cone attached to a distal end of the elongated catheter body.

According to embodiments of the present invention the stent is advanced forward against the nose cone.

According to embodiments of the present invention pulling back the outer catheter tube to thereby deploy the stent disengages it from the stent holder.

According to embodiments of the present invention the distal portion is 20-80 mm in length.

According to embodiments of the present invention the distal portion has an elasticity of 170-513 mPa.

According to embodiments of the present invention the catheter further comprises a handle for activating the first and the second mechanism.

According to one aspect of the present invention there is provided a method of delivering a stent through a tortuous blood vessel comprising advancing a catheter having a distal portion that is more elastic than a proximally adjacent portion through the tortuous blood vessel, the catheter carrying a stent within the proximally adjacent portion; advancing the stent forward into the distal portion and deploying the stent.

According to embodiments of the present invention the catheter includes an elongated catheter body having an outer catheter tube surrounding an inner catheter tube.

According to embodiments of the present invention the inner catheter tube includes a guidewire lumen.

According to embodiments of the present invention the guidewire lumen extends from a first opening at a distal tip of the elongated catheter body to a second opening at a side wall of the outer catheter tube.

According to embodiments of the present invention the catheter further comprises a nose cone attached to a distal end of the elongated catheter body.

According to embodiments of the present invention the stent is advanced forward against the nose cone.

According to embodiments of the present invention deploying the stent is effected by pulling back the outer catheter tube.

According to embodiments of the present invention the distal portion is 20-80 mm in length.

According to embodiments of the present invention the distal portion has an elasticity of 170-513 mPa.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE SEVERAL VIEWS OF THE DRAWING(S)

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1A illustrates an embodiment of the present delivery catheter having a standard over-the-wire configuration.

FIGS. 1B-E illustrate advancement and deployment of a stent carried by the delivery catheter of FIG. 1A.

FIG. 2A-C illustrate the present catheter connected to a handle with the slider button in stent preloaded position (FIG. 2A) and forward deployed position (FIG. 2B) and the y-connector in a pulled-back position to unsheathe stent (FIG. 2C).

FIG. 2D illustrates the internal components of the handle shown in FIGS. 2A-C.

FIGS. 2E-G illustrate a second embodiment of a handle.

FIG. 3 illustrates the guidewire lumen and stent advancement mechanism of the rapid exchange embodiment of the present delivery catheter.

FIGS. 4A-D illustrate advancement and deployment of a stent carried by the rapid exchange embodiment of the present delivery catheter.

FIG. 5 schematically illustrates a stent constructed in accordance with the teachings of the present invention and deliverable using the present catheter system.

FIG. 6 is a prototype stent constructed in accordance with the teachings of the present invention.

### DETAILED DESCRIPTION

The present invention is of a delivery catheter which can be used to deliver a deployable device through tortuous anatomy. Specifically, the present invention can be used to deliver a stent to a site at or past tortuous anatomy.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Catheters for deploying devices such as stents in the vasculature typically carry such devices at or near the distal end of the catheter. Once positioned in place, the stent is either unsheated for self-deployment or is expanded via balloon inflation.

While maneuvering such catheters in non-tortuous vasculature is straightforward, tortuous vasculature can present a challenge due to the fact that the distally-mounted stent substantially increases the stiffness of the catheter distal portion and thus reduces maneuverability.

While reducing the present invention to practice, the present inventor devised a catheter system that can be easily maneuvered through tortuous vessels and yet can deliver a stent or any other deployable device to a body vessel site at or past the tortuous vessel.

Thus, according to one aspect of the present invention there is provided a catheter for delivering a deployable device such as a stent into a body vessel.

As used herein the phrase “deployable device” refers to any device that can be deployed from a delivery catheter at an anatomical location such as a lumen of a blood vessel. Examples include stents (including stent grafts), coils and the like.

As used herein, the phrase “body vessel” refers to any vessel or conduit inside the body. Examples include blood vessels (e.g., artery, vein), urinary tract vessels, the spinal column, fallopian tubes, bile ducts, and the like.

The present catheter includes an elongated catheter body having an outer catheter tube surrounding an inner catheter tube. The elongated catheter body is attached at a proximal end to a handle and a nose cone at the distal end. The elongated catheter body includes a distal portion that is more elastic than a proximally adjacent portion of the elongated catheter body. Such a distal portion can be 20-80 mm in length with an elastic modulus of 12-170 mPa (Such as Pebax 2533 or Pebax 5533 or any other polymer with such durometer). The elastic/flexible portion of the catheter body facilitates navigation through tortuous vessels without damaging vessel walls.

The rest of the catheter body including the proximally adjacent portion can be 20-2000 mm in length with an elastic modulus of 170-513 mPa. (Such as Pebax 5533 or Pebax 7233 or any other polymer with such durometer). The external diameter of the elongated catheter body can be 1.0-8.0 mm.

The outer catheter tube can have an internal diameter of 0.8-7.8 mm and the inner catheter tube can have an external diameter of 0.5-2 mm and an internal diameter of 0.3-1.8 mm. The inner catheter tube can run the length of catheter body or to an opening along the length of the outer catheter tube (100-400 mm from the distal end of the outer tube). The lumen of the inner catheter tube serves as a guidewire lumen in a standard over-the-wire configuration or a rapid exchange configuration.

The present catheter can also include a holder for advancing and optionally holding the deployable device (e.g., stent) within the outer catheter tube and around the inner catheter tube at the proximally adjacent portion. Thus, when the catheter is advanced from an access site to the target anatomy, the present catheter maintains the stent proximally to the flexible distal portion of the catheter body (at the proximally adjacent portion) and enables navigation through tortuous anatomy.

The present catheter can further include a first mechanism for advancing the stent forward into the distal portion (and against the nose cone). Such a mechanism is deployable from the handle and includes a pushrod/wire (within a sleeve) running from the handle actuation mechanism to the stent holder.

The present catheter can further include a second mechanism for pulling back the outer catheter tube (against the inner catheter tube) to thereby deploy the stent. Such a mechanism is deployable from the handle and includes a handle actuation mechanism that is attached to the outer catheter tube. Deployment of the stent (radial expansion) disengages the stent from the stent holder (if the stent is engaged thereto) and enables delivery thereof to the vessel lumen.

The rapid exchange configuration of the present catheter requires two adjacent lumens to move one relative to the other within an external surrounding lumen. In addition, the guidewire lumen is concentric distally to the stent holder in order to allow stent loading (advancing forward).

As is mentioned herein, the present catheter is particularly suitable for delivery of deployable devices through tortuous anatomy. One such deployable device is a stent that can be delivered through tortuous blood vessels of the brain (e.g., veins) for the purpose of dilating strictures.

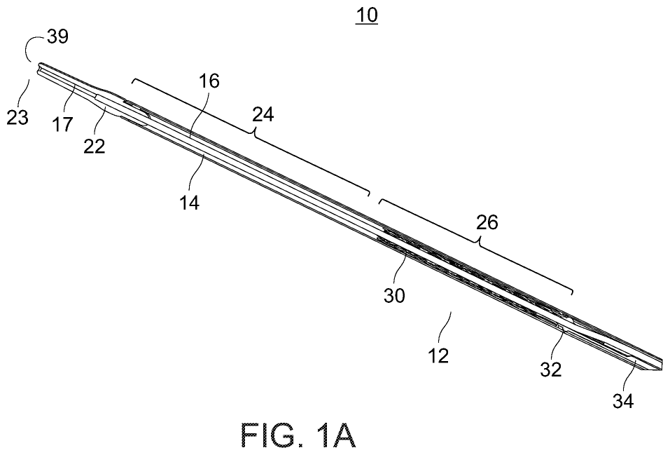

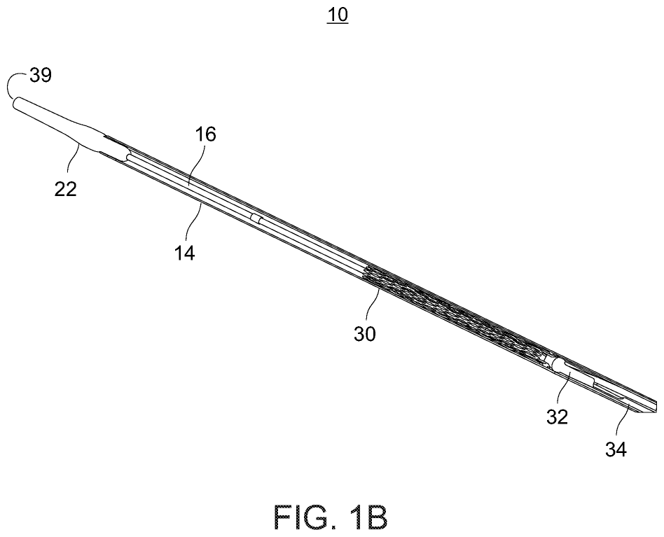

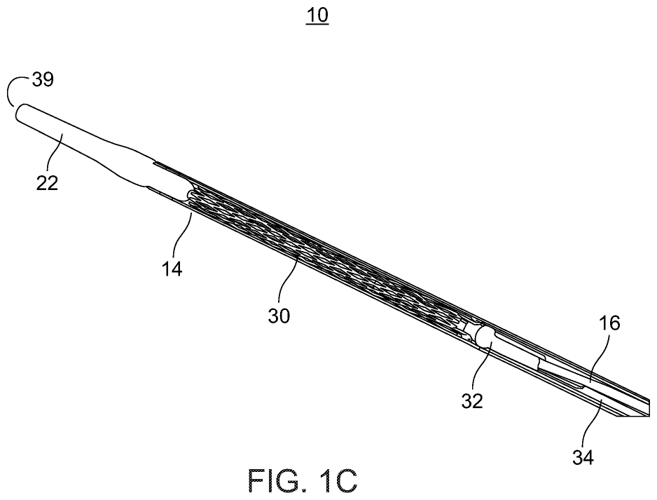

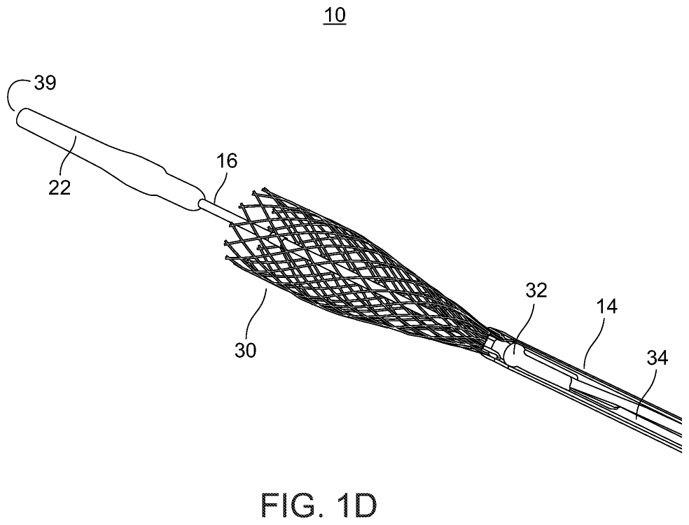

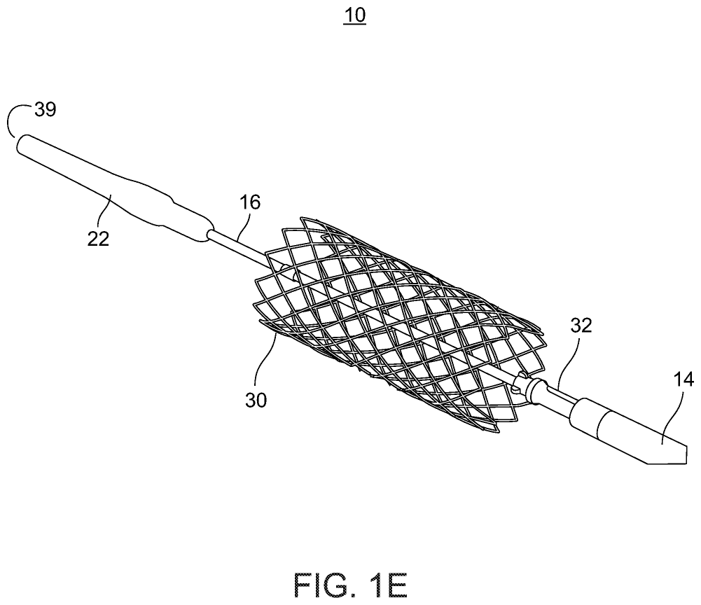

Referring now to the drawings, FIGS. 1-4D illustrate several embodiments of a catheter (referred to hereinunder as catheter 10) suitable for delivering a device such as a stent to a location in the vasculature system.

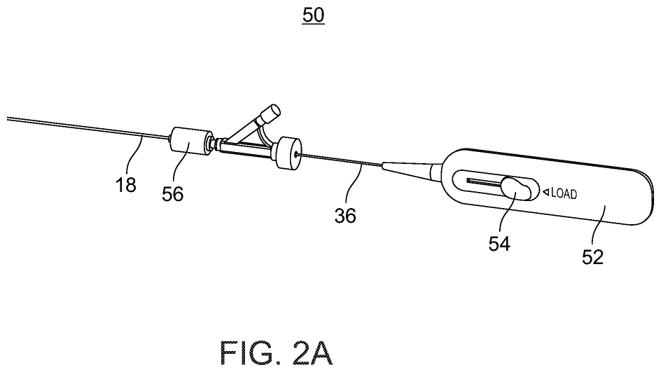

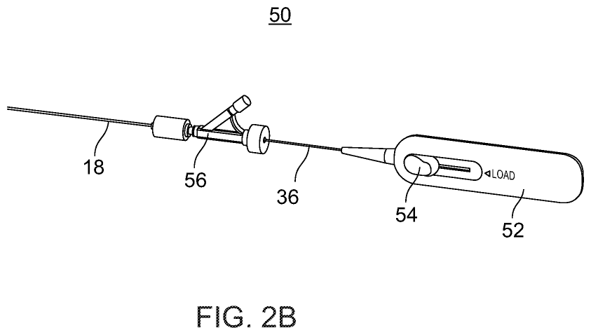

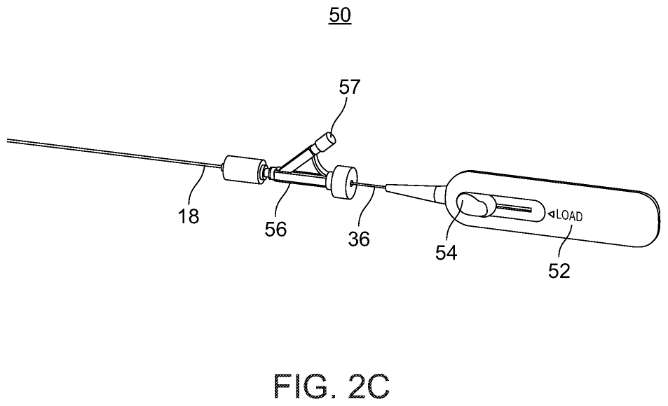

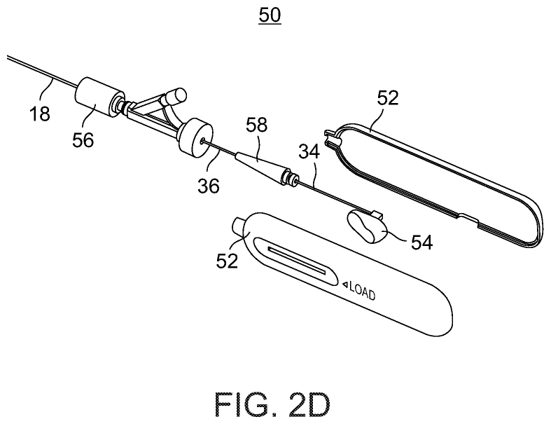

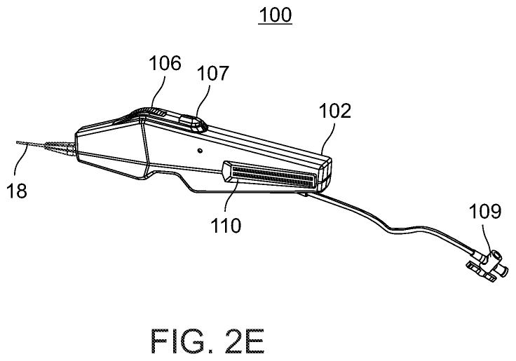

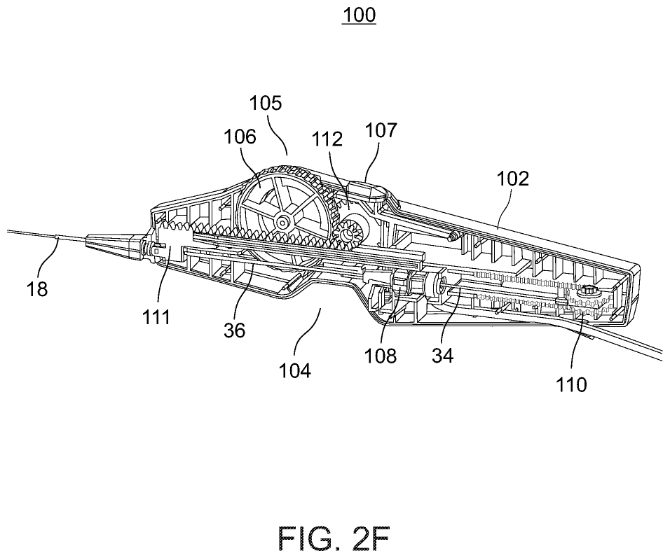

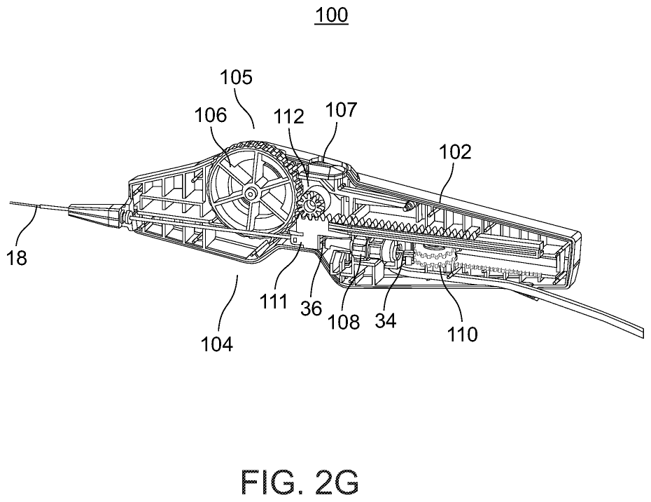

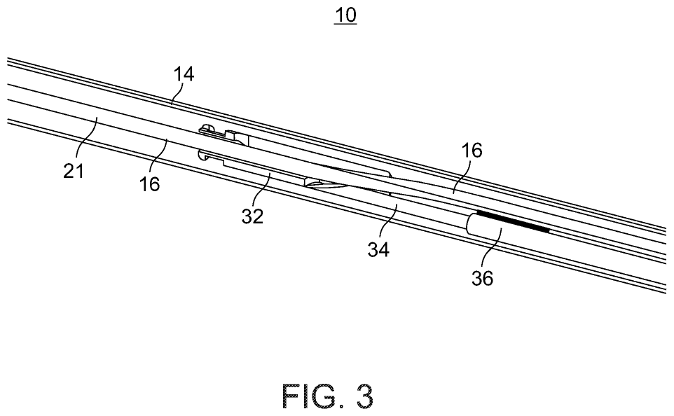

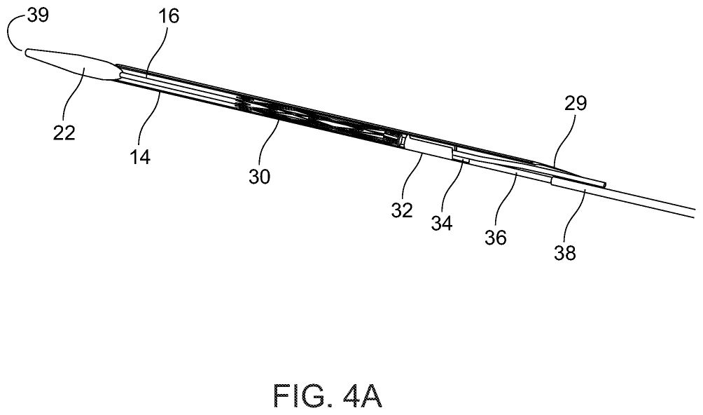

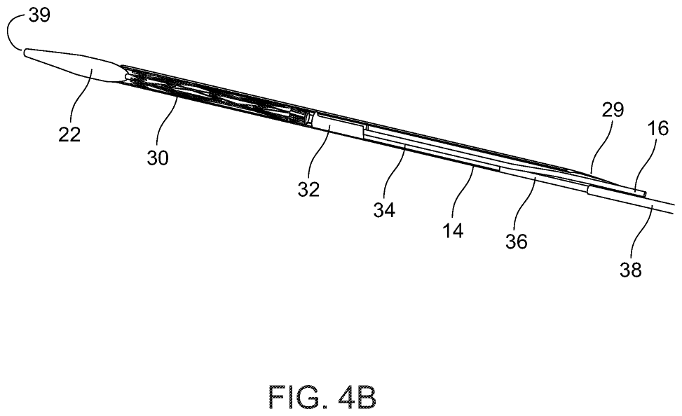

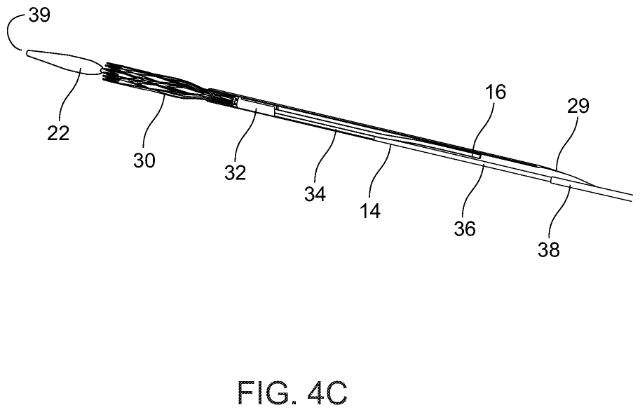

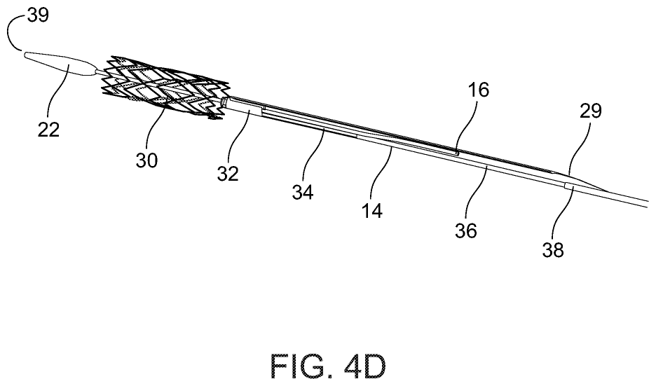

FIGS. 1A-E illustrate an over-the-wire configuration of catheter 10 whereas FIGS. 3 and 4A-D illustrate a rapid exchange configuration of catheter 10. FIGS. 2A-D illustrates a handle that can be used with the rapid exchange configuration of catheter 10.

Catheter 10 includes an elongated catheter body 12 having an outer catheter tube 14 surrounding an inner catheter tube 16. The lumen of inner catheter tube 16 serves as a guidewire 17 lumen. Elongated catheter body 12 is attached at a proximal end 18 to a handle 50 (FIG. 2) and a nose cone 22 at the distal end 23. Elongated catheter body 12 can include a distal portion 24 that is more elastic than a proximally adjacent portion 26 although a catheter 10 having an elongated catheter body 12 with a unitary elasticity throughout its length is also envisaged herein. Distal portion 24 of catheter body 12 can facilitate navigation through tortuous vessels without damaging vessel walls.

Elongated catheter body 12 is configured for carrying a stent 30 within outer catheter tube 14 and over inner catheter tube 16 at proximally adjacent portion 26. Stent 30 can be a self-deploying stent or a balloon expanded stent, a self-deploying stent is shown in the Figures. Stent 30 can be 15-80 mm in length, 1-8 mm in diameter (when packed within outer tube 14, FIG. 1A) and 2-24 mm in diameter when expanded (as is shown in FIG. 1E). Stent 30 of the configuration shown in the Figures is designed for deployment within brain blood vessels for the purpose of treating stenosis or strictures.

Catheter 10 further includes a stent holder 32 for advancing the stent forward. The stent holder can also engage the stent via, for example, radial protrusions for engaging eyelets formed within proximal struts of stent 30. Expansion of stent 30 (FIG. 1E) caused by unsheathing (pulling back outer catheter tube 14, as is further described hereinbelow) releases the eyelets from the radial protrusions and disengages stent 30 from holder 32.

Holder 32 is movable over inner catheter tube 16 (and within outer catheter tube 14) via a wire/rod 34 that is connected to a first mechanism within the handle (further described below with reference to FIGS. 2A-D). Holder can be advanced to move stent 30 forward into distal portion 24 and retracted to pull stent 30 back into proximally adjacent portion 26 if need be (e.g., to abort deployment or reposition catheter 10).

Outer catheter tube 14 is connected to a second mechanism within the handle that actuates unsheathing of stent 30 by pulling back outer catheter tube 14 with respect to inner catheter tube 16. Inner catheter tube 16 is connected to mid tube 36 which connects to handle body 52 (FIG. 2A).

Catheter 10 is navigated over-the-wire through the blood vessel with stent 30 carried within proximally adjacent portion 26 (FIG. 1B). Once in position, stent 30 is advanced to distal portion 24 against nose cone 22 (FIG. 1C, outer catheter tube shown in section view for clarity) and outer catheter tube 14 is pulled back using the second handle mechanism to allow stent 30 to expand (FIG. 1D-E). Such expansion releases stent 30 from holder 32 to thereby deploy it at the site of treatment. Outer catheter tube 14 can then be advanced forward to cover distal portion 24 and catheter 10 can be removed from the body.

FIGS. 2A-D illustrate a handle (referred to herein as handle 50) that can be used with catheter 10.

Handle 50 includes a handle body 52 (formed from two halves) that houses a first mechanism 54 for advancing and retracting stent 30, and a second mechanism 56 (Y-connector 56) for longitudinally translating outer catheter tube 14 with respect to inner catheter tube 16. Y-connector 56 can also include an irrigation port 57. Outer Catheter tube 16 is connected to mid catheter tube 36. Tube 36 is fixed to handle 52 via cone 58 (FIG. 2D) with outer catheter tube 14 and holder 32 being movable relative to catheter tube 36.

FIG. 2A illustrates the initial handle position in which button/slider advancement mechanism 54 is in a proximal position and Y-connector 56 is in a distal position relative to handle 52. Button/slider advancement mechanism 54 is connected to wire\rod 34.

To deploy stent 30, advancement mechanism 54 is moved distally (FIG. 2B) to advance stent 30 to distal portion 24. Stent 30 is then unsheathed by pulling Y-connector 56 (proximally) relative to handle 52 (FIG. 2C). At this position, stent 30 is fully unsheathed and is free to self-expand and release from holder\pusher 32.

FIGS. 2E-G illustrates another embodiment of handle 100 that can be used with catheter 10. Handle 100 includes a handle body 102 and a ratchet-like advancement mechanism 104 (FIG. 2E). FIGS. 2F-G illustrate the internal components of handle 100 and in particular advancement mechanism 104.

Handle 100 includes a handle body 102 (formed from two halves) that houses mechanism 104 for advancing and retracting stent 30, and a gear mechanism 105 that includes knob 106, gear 112 and rack 111 for longitudinally translating outer catheter tube 14 with respect to inner catheter tube 36. Mechanism 104 also includes an irrigation port 109. Outer Catheter tube 14 is connected to catheter tube 18. Tube 36 is fixed to handle 102 via flush port 108 (FIGS. 2F-G) with outer catheter tube 18 and holder 32 being movable relative to catheter tube 36. Mechanism 104 also include a loading advancement actuator 110 that is connected to wire/rod 34 and a release button 107 that prevents premature release of stent 30.

FIG. 2F illustrates an initial handle position in which loading/advancement actuator 110 is in a proximal position and rack 111 is in a distal position relative to handle 102. Actuator 110 is connected to wire/rod 34.

To deploy stent 30, actuator 110 is moved distally (FIG. 2G) to advance stent 30 to distal portion 24. Stent 30 is then unsheathed by turning knob 106 (clockwise) relative to handle 102 (FIG. 2G). At this position, stent 30 is fully unsheathed and is free to self-expand and release from holder/pusher 32.

FIGS. 3 and 4A-D illustrate a rapid exchange configuration of catheter 10. Catheter 10 includes an elongated catheter body 12, an outer catheter tube 14 and an inner catheter tube 16. Inner catheter tube 16 does not extend the length of elongated catheter body 12 (as is the case with the configuration shown in FIGS. 1A-E) but rather extends from nose cone 22 to an opening 29 (FIG. 4A) along a length of outer catheter tube 14 (100-300 mm from distal end 23). A lumen 21 of inner catheter tube serves as a guidewire lumen and as such, the guidewire inserts at nose cone 22 opening 39 and exits at opening 29.

As with the standard configuration, wire/rod 34 translates holder 32 and stent 30 carried thereby over inner tube 16 to move stent 30 from proximally adjacent portion 26 and into distal portion 24 (FIG. 4B) following catheter navigation and prior to stent 30 deployment (FIGS. 4C-D).

The rapid exchange configuration of catheter 10 provides several advantages including:
(i) Improved pushability to help cross lesions or strictures;
(ii) Reduced fluoroscopy times due to faster guidewire threading;
(iii) Enables a single operator to maintain wire position; and
(iv) Easier device exchanges with shorter guidewires.

Catheter 10 of the present invention can be used in a cerebral vein, cerebral arteries, Renal arteries and veins, coronary arteries or any other suitable vessels. The advantage of the system is its ability to cross through a tortuous anatomy. The guidewire is held fixed in position and catheter 10 is advanced over the wire to position the distal portion of catheter 10 at the site of treatment. Stent 30 is then advanced within catheter 14 to distal section 24 (FIGS. 1C, 4B) by pushing distally deployment mechanism 54 (FIG. 2B). Y-connector 56 is then pulled proximally towards handle body 52 (FIG. 2C) to pull back outer tube 14 and unsheath stent 30, allowing it to expand. Once stent 30 is fully exposed it releases from holder 32. The catheter is then pulled out of the blood vessel.

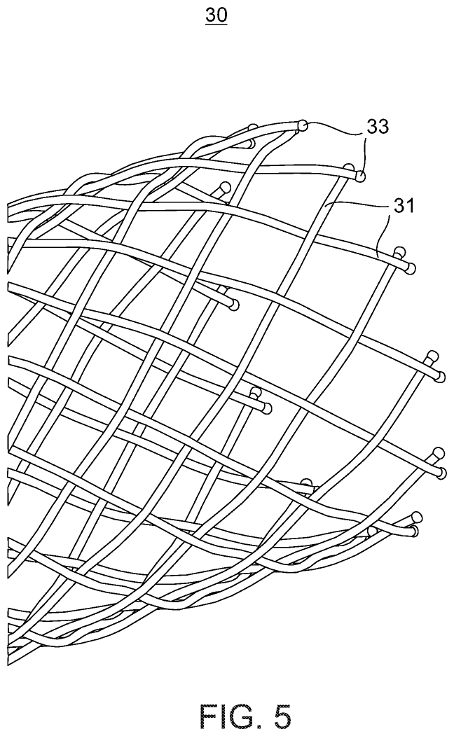

As is mentioned herein, delivery catheter 10 can be used to deliver any stent 30. Preferred are stents that have an open braid architecture since such stents possess superior kink resistance and can be easily re-sheathed. As is described in Example 1 of the Examples section that follows, while testing prototypes of the present invention, the present inventor realized that open braid stents can snag on the delivery sheath (outer catheter tube 14) when pushed distally during loading. In order to overcome this limitation of open braid stents, the present inventor treated the ends of wires 31 forming the braid of stent 30 (as is shown in FIG. 5) to form ball-like protrusions 33 so as to reduce the likelihood of such snagging.

As used herein the term “about” refers to ±10%.

Additional objects, advantages, and novel features of the present invention will become apparent to one ordinarily skilled in the art upon examination of the following examples, which are not intended to be limiting.

### EXAMPLES

Reference is now made to the following examples, which together with the above descriptions, illustrate the invention in a non-limiting fashion.

### Example 1

### Modified Stent

Testing the stent loading mechanism of the present invention using an in-vitro test model revealed that when stent loading was performed on a linear catheter loading was smooth and did not require the operator to apply much force. However, when the distal end of the catheter was bent at a relatively small radius (10-20 mm), operators observed that loading required more force. Through these experiments the present inventor observed that the exposed braid ends (that are mechanically cut) are very sharp and tend to snag against the inner soft PTFE layer of the catheter outer tube. To remedy this problem, the present inventor utilized a laser welding machine to melt the wire ends and form a ball-like protrusions that eliminated the snagging phenomena previously observed.

### Example 2

### Animal Studies

A prototype of the present delivery catheter and an off-the-shelf delivery catheter (Cordis Pro-Rx stent system) were used to deliver a stent to the internal mammary vein of pigs; ease of delivery and histological impact on vein tissue were assessed for both catheters.

### Procedure

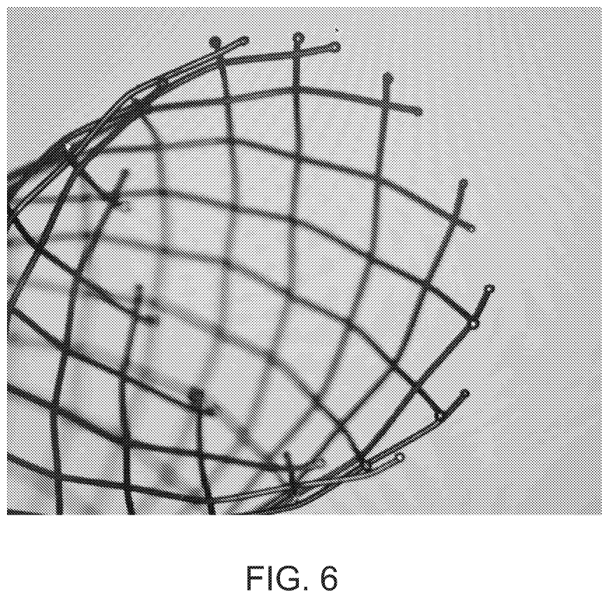

Each animal was placed in dorsal recumbency and a Hartmann solution (1 Liter×2) was administered IV via constant rate infusion. An introducer sheath was placed in a femoral artery for blood pressure and vital signs measurements. Heparin was administered IV to maintain activated clotting time (ACT) of 250-350 sec, throughout the procedure. An introducer sheath of an appropriate size was placed in the femoral vein and a pigtail/multipurpose catheter was inserted over a guidewire, through the sheath in the femoral vein and a radiopaque solution was injected to perform fluoroscopic angiography.
The off-the-shelf delivery catheter loaded with the stent (Cordis Pro RX 8×40) was advanced through the introducer sheath in the femoral vein, over the guidewire, to the right or left internal mammary vein; the stent was then self-expanded via unsheathing. The vein diameter at the selected site for implantation was measured and recorded prior to implantation and was in the range of 3-7 mm.
The present catheter was used to deliver the new stent design shown in FIG. 6 (7×50) into a pig mammary vein using the procedure described above. The vein diameter at the selected site for implantation was measured and recorded prior to implantation and was in the range of 3-7 mm.

### Results and Conclusions

During the advancement to the right mammary vein, it was observed that the off-the-shelf catheter, advanced over a wire, could not bend and tolerate small radiuses. An attempt to push a guiding catheter into the mammary vain in order to guide the catheter tip also failed due to a small vein diameter.
The present catheter was advanced over the same wire and easily navigated through the narrow opening into the mammary vein. The flexible distal portion of present catheter showed superior maneuverability when compared to the off-the-shelf catheter and did not require a larger guiding catheter to reach the Mammary vein.
The stent delivered by the present catheter and the stent delivered by the off-the-shelf catheter were maintained in the animals for two weeks and then sent to histopathology. The new stent design showed similar histopathology results to the off the shelf stent.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A catheter for stent delivery comprising:
(a) an elongated catheter body having an outer catheter tube surrounding an inner catheter tube;
(b) a stent holder for holding a stent within said outer catheter tube at a portion proximally adjacent to a distal portion;
(c) a first mechanism for advancing said stent forward into said distal portion; and
(d) a second mechanism for pulling back said outer catheter tube to thereby deploy said stent.

2. The catheter of claim 1, wherein said distal portion is more elastic than said portion proximally adjacent to said distal portion of said elongated catheter body.

3. The catheter of claim 1, wherein said inner catheter tube includes a guidewire lumen.

4. The catheter of claim 1, wherein said guidewire lumen extends from a first opening at a distal tip of said elongated catheter body to a second opening at a side wall of said outer catheter tube.

5. The catheter of claim 1, further comprising a nose cone attached to a distal end of said elongated catheter body.

6. The catheter of claim 5, wherein said stent is advanced forward against said nose cone.

7. The catheter of claim 1, wherein pulling back said outer catheter tube to thereby deploy said stent disengages it from said stent holder.

8. The catheter of claim 1, wherein said distal portion is 20-80 mm in length.

9. The catheter of claim 1, wherein said distal portion has an elasticity of 170-513 mPa.

10. The catheter of claim 1, further comprising a handle for activating said first and said second mechanism.

11. A method of delivering a stent through a tortuous blood vessel comprising:
(a) advancing a catheter through the tortuous blood vessel, said catheter carrying a stent within a portion proximally adjacent to a distal portion;
(b) advancing said stent forward into said distal portion; and
(c) deploying said stent.

12. The method of claim 11, wherein said distal portion is more elastic than said portion proximally adjacent to said distal portion of said elongated catheter body.

13. The method of claim 11, wherein said catheter includes an elongated catheter body having an outer catheter tube surrounding an inner catheter tube.

14. The method of claim 13, wherein said inner catheter tube includes a guidewire lumen.

15. The method of claim 14, wherein said guidewire lumen extends from a first opening at a distal tip of said elongated catheter body to a second opening at a side wall of said outer catheter tube.

16. The method of claim 13, wherein said elongated catheter body further comprising a nose cone attached to a distal end thereof.

17. The method of claim 16, wherein said stent is advanced forward against said nose cone.

18. The method of claim 13, wherein (d) is carried out by pulling back said outer catheter tube.

19. The method of claim 11, wherein said distal portion is 20-80 mm in length, or wherein said distal portion has an elasticity of 170-513 mPa.

20. (canceled)

21. A stent comprising a tubular braid having exposed wire ends, wherein each of said ends includes a ball-like protrusion.
